(19) United States
(12) Patent Application Publication
(10) Pub. No.: US 2006/0177388 A1
(43) Pub. Date: Aug. 10, 2006

(54) Tanning aids with a high protection factor

(76) Inventors: Kai-Oliver Schocke, Darmstadt (DE); Peter Seelmann, Vienna (AT); Uwe Hild, Gross-Umstadt (DE); Birgit Haefner, Muenster (DE); Gerald Molnar, Gramatsneusiedl (AT); Erich Auer, Gramatsneusiedl (AT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/546,804

(22) PCT Filed: Mar. 2, 2004
(86) PCT No.: PCT/EP04/02064

§ 371 Date: Aug. 25, 2005

(30) Foreign Application Priority Data
May 3, 2003 (DE) 10320810.0

(51) Int. Cl. A61K 8/00

(52) U.S. Cl. 424/59

(57) ABSTRACT

The present invention relates to tanning aids, comprises a polymethyl methacrylate shaped body, where the transparency of the polymethyl methacrylate shaped body at 380 nm is at least 40%, and the polymethyl methacrylate shaped body comprises 0.005 to 0.1% by weight, based on the weight of the polymethyl methacrylate shaped body, of at least one triazine compound according to formula (I) 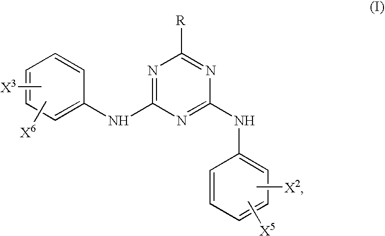 in which
R is halogen, C₁-C₂₂-alkyl, C₁-C₂₂-alkoxy, C_{I}-C₂₂-hydroxyalkoxy, C_{I}-C₂₂-alkoxyalkyl, —NHR₁—, —N(R¹)₂, or a radical of the formula (Ia) 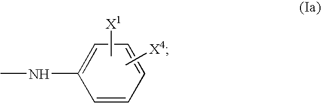
X¹, X² and X³,
   independently of one another, are —CONHR¹, —NR²R³, —SO₂R⁴, —CN, —COR⁵ or —COOR⁶,
   R¹ is hydrogen, C₁-C₂₂-alkyl, C₆-C₂₀-aryl, C₇-C₂₀-aralkyl, C₅-C₈-cycloalkyl, or a radical of the formula (Ib) 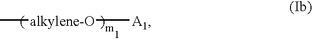 in which
      A₁ is C_{I}-C₈-alkyl, C₅-C₆-cycloalkyl, C₆-C₂₀-aryl, C₇-C₂₀-aralkyl; and
      m₁ is 1 to 10,
   R² and R³, independently of one another, are hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —COR¹;
   R⁴ is hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —NR²R³;
   R⁵ and R⁶, independently of one another, are hydrogen, C_{I}-C₂₂-alkyl, C₆-C₈-cycloalkyl, C₆-C₂₀-aryl, C₇-C₂₀-aralkyl or a radical of the formula (Ib);
X⁴, X⁵ and X⁶, independently of one another, are hydrogen or hydroxyl.

The present invention relates to tanning aids with a high protection factor which comprise a polymethyl methacrylate shaped body.

A slightly tanned skin is a sign of holiday and health. In order to tan the skin, sunscreen creams and the like are usually used as tanning aids in order to protect the skin against damage by UV radiation. A disadvantage of these auxiliaries is that such creams can trigger allergies in sensitive people. Furthermore, many of these substances are not water-resistant. Accordingly, they are removed during bathing, and are then not applied again. This negligence can easily lead to skin damage.

Moreover, devices are known which bring about tanning of the skin with the help of incorporated UV emitters. Also known in this connection are lying devices for solaria applications made of PMMA, which comprise large amounts of UV stabilizers and/or UV absorbers in order to protect the plastic against degradation by UV radiation. Tanning with sunlight is not possible here, however. A disadvantage of such devices is the high energy consumption of the UV emitters. Moreover, these installations are not intended for operation outdoors, meaning that tanning is more likely to be perceived as boring.

In view of the prior art given and discussed here, it was consequently an object of the present invention to provide tanning aids which can be used to achieve natural tanning of the skin using sunlight without the skin coming into contact with sunscreen cream.

Moreover, it was an object of the present invention to provide a tanning aid which is particularly easy to maintain.

A further object of the invention was that the tanning aids have high durability, in particular high resistance to UV irradiation or weathering.

Furthermore, the object underlying the invention was to provide tanning aids which can be produced in a particularly simple manner. Thus, for the production of the tanning aids, it should be possible, in particular, to use substrates which are obtainable by extrusion, injection moulding, and by casting processes.

A further object of the present invention was to provide tanning aids whose sun protection can be adjusted in a particularly simple manner. For example, it should be possible to adjust the time spent under the tanning aid to a pregiven time for many skin types. With regard to classical sunscreen cream, it should be possible to adjust these tanning aids accordingly to a certain sun protection factor.

Moreover, it should be possible to adapt the tanning aids of the present invention in a particularly simple manner to different requirements and applications. Thus, for example, portable screens and built-in roofs should be available. In this connection, it should be possible to adapt the tanning aids to the requirements in a simple manner with respect to size and shape.

These objects and others which, while not specified in terms of words, can be derived quite naturally from the connections discussed herein or arise automatically from these, are achieved by the tanning aids described in claim 1.

Advantageous modifications of the tanning aids according to the invention are protected in the dependent claims which relate back to claim 1.

As a result of the fact that the tanning aid comprises a polymethyl methacrylate shaped body, where the transparency of the polymethyl methacrylate shaped body at 380 nm is at least 40%, and the polymethyl methacrylate shaped body comprises 0.005 to 0.1% by weight, based on the weight of the polymethyl methacrylate shaped body, of at least one triazine compound according to formula (I)

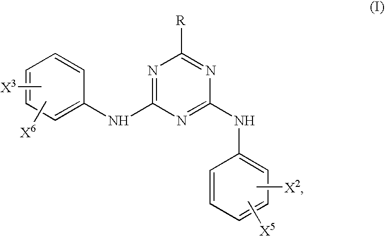

in which
R is halogen, C₁-C₂₂-alkyl, C_{I}-C₂₂-alkoxy, C_{I}-C₂₂-hydroxyalkoxy, C_{I}-C₂₂-alkoxyalkyl, —NHR₁—, —N(R¹)₂, or a radical of the formula (Ia) 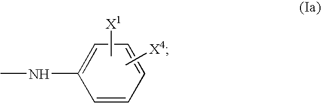
X¹, X² and X³,
   independently of one another, are —CONHR¹, —NR²R³, —SO₂R⁴, —CN, —COR⁵ or —COOR⁶, in which
   R¹ is hydrogen, C₁-C₂₂-alkyl, C₆-C₂₀-aryl, C₇-C₂₀-aralkyl, C₅-C₈-cycloalkyl, or a radical of the formula (Ib) 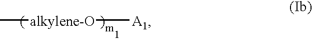
      in which
      A₁ is C_{I}-C₈-alkyl, C₅-C₆-cycloalkyl, C₆-C₂₀-aryl, C₇-C₂₀-aralkyl;
      and
      m₁ is 1 to 10,
   R² and R³, independently of one another, are hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —COR¹ ;
   R⁴ is hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —NR²R³;
   R⁵ and R⁶, independently of one another, are hydrogen, C_{I}-C₂₂-alkyl, C₆-C₈-cycloalkyl, C₆-C₂₀-aryl, C₇-C₂₀-aralkyl or a radical of the formula (Ib);
X⁴, X⁵ and X⁶, independently of one another, are hydrogen or hydroxyl,

it is surprisingly possible to provide a tanning aid with the help of which natural tanning with sunlight is possible without the skin coming into contact with sunscreen cream.

The measures according to the invention achieve, inter alia, in particular the following advantages:
The tanning aids according to the invention make tanning outside possible.
Furthermore, it is possible to dispense with energy-intensive UV emitters during tanning.
The tanning aids according to the invention are easy to maintain and easy to produce. Thus, it is possible, in particular, to use shaped bodies which are obtainable by extrusion, injection moulding and casting processes.
The tanning aids are weathering-resistant and, in particular, resistant to UV radiation. Furthermore, the tanning aids according to the invention have very good mechanical properties.
Furthermore, the tanning aids according to the invention can be adapted easily to very diverse requirements. Thus, it is possible to produce, in particular, transportable screens or solid roofs in any size in order to permit natural tanning of the skin without having to worry about skin damage as a result.
Moreover, the tanning aids can be produced for each skin type, meaning that different durations outside can be provided.

For the purposes of the present invention, the term tanning aid means a device which comprises at least one polymethyl methacrylate shaped body which can be brought between the sunlight and a skin surface to be tanned. Accordingly, these may, in particular, be transparent roofs of buildings or screens which are, for example, immovably fixed. Moreover, roofs which can be fixed to boats, in particular paddle boats, electric boats and the like, are suitable as tanning aids.

Moreover, however, they may also be transportable screens which, depending on the thickness of the polymethyl methacrylate shaped body, are fixed in terms of their dimensions, or which can also be collapsed and stored.

The tanning aid according to the invention comprises a polymethyl methacrylate shaped body. Polymethyl methacrylate (PMMA) is known per se in the specialist field. The polymethyl methacrylate shaped body preferably comprises at least 30% by weight, based on the weight of the polymethyl methacrylate shaped body, of polymethyl methacrylate.

Polymethyl methacrylates are generally obtained by free-radical polymerization of mixtures which comprise methyl methacrylate. In general, these mixtures comprise at least 40% by weight, preferably at least 60% by weight and particularly preferably at least 80% by weight, based on the weight of the monomers, of methyl methacylate.

In addition, these mixtures for the preparation of polymethyl methacrylates can comprise further (meth)acrylates which are copolymerizable with methyl methacrylate. The expression (meth)acrylate includes methacrylates and acrylates, and mixtures of the two.

These monomers are also known. These include, inter alia, (meth)acrylates, which are derived from saturated alcohols, such as, for example, methyl acrylate, ethyl(meth)acrylate, propyl(meth)acrylate, n-butyl(meth)acrylate, isobutyl(meth)acrylate tert-butyl(meth)acrylate, pentyl(meth)acrylate and 2-ethylhexyl(meth)acrylate;

(meth)acrylates which are derived from unsaturated alcohols, such as, for example, oleyl(meth)acrylate, 2-propynyl(meth)acrylate, allyl(meth)acrylate, vinyl(meth)acrylate;

Aryl(meth)acrylates, such as benzyl(meth)acrylate or phenyl(meth)acrylate, where the aryl radicals may in each case be unsubstituted or substituted up to four times;

cycloalkyl (meth)acrylates, such as 2-vinylcyclohexyl(meth)acrylate, bornyl(meth)acrylate;

hydroxyalkyl(meth)acrylates, such as 3-hydroxypropyl(meth)acrylate, 3,4-dihydroxybutyl(meth)acrylate, 2-hydroxyethyl(meth)acrylate, 2-hydroxypropyl(meth)acrylate;

glycol di(meth)acrylates, such as 1,4-butandiol(meth)acrylate, (meth)acrylates of ether alcohols, such as tetrahydrofurfuryl(meth)acrylate, vinyloxyethoxyethyl(meth)acrylate;

amides and nitriles of (meth)acrylic acid, such as N-(3-dimethylaminopropyl)(meth)acrylamide, N-(diethylphosphono)(meth)acrylamide, 1-methacryloylamido-2-methyl-2-propanol; sulphur-containing methacrylates, such as ethylsulphinylethyl(meth)acrylate, 4-thiocyanatobutyl(meth)acrylate, ethylsulphonylethyl(meth)acrylate, thiocyanatomethyl(meth)acrylate, methylsulphinylmethyl(meth)acrylate, bis((meth)acryloyloxyethyl)sulphide;

polyvalent(meth)acrylates, such as trimethyloylpropane tri(meth)acrylate.

As well as the (meth)acrylates listed above, the compositions to be polymerized can also have further unsaturated monomers which are copolymerizable with methyl methacrylate and the abovementioned (meth)acrylates.

These include, inter alia, 1-alkenes, such as 1-hexene, 1-heptene; branched alkenes, such as, for example, vinylcyclohexane, 3,3-dimethyl-1-propene, 3-methyl-1-diisobutylene, 4-methyl-1-pentene;

acrylonitriles; vinyl esters, such as vinyl acetate; styrene, substituted styrenes with an alkyl substituent in the side chain, such as, for example, α-methylstyrene and α-ethylstyrene, substituted styrenes with an alkyl substituent on the ring, such as vinyltoluene and p-methylstyrene, halogenated styrenes, such as, for example, monochlorostyrenes, dichlorostyrenes, tribromostyrenes and tetrabromostyrenes;

heterocyclic vinyl compounds, such as 2-vinylpyridine, 3-vinylpyridine, 2-methyl-5-vinylpyridine, 3-ethyl-4-vinylpyridine, 2,3-dimethyl-5-vinylpyridine, vinylpyridimidine, vinylpiperidine, 9-vinylcarbazole, 3-vinylcarbazole, 4-vinylcarbazole, 1-vinylimidazole, 2-methyl-l-vinylimidazole, N-vinylpyrrolidone, 2-vinylpyrrolidone, N-vinylpyrrolidine, 3-vinylpyrrolidine, N-vinylcaprolactam, N-vinylbutyrolactam, vinyloxolane, vinylfuran, vinylthiophene, vinylthiolan, vinylthiazoles and hydrogenated vinylthiazoles, vinyloxazoles and hydrogenated vinyloxazoles;

vinyl and isoprenyl ethers;

maleic acid derivatives, such as, for example, maleic anhydride, methylmaleic anhydride, maleinimide, methylmaleinimide; and dienes, such as, for example, divinylbenzene.

In general, these comonomers are used in an amount of from 0 to 60% by weight, preferably 0 to 40% by weight and particularly preferably 0 to 20% by weight, based on the weight of the monomers, where the compounds can be used individually or as a mixture.

The polymerization is generally started using known free-radical initiators. Preferred initiators include, inter alia, the azo initiators known throughout the specialist field, such as AIBN and 1,1-azobiscyclohexanecarbonitrile, and peroxy compounds, such as methyl ethyl ketone peroxide, acetylacetone peroxide, dilauryl peroxide, tert-butyl per-2-ethylhexanoate, ketone peroxide, methyl isobutyl ketone peroxide, cyclohexanone peroxide, dibenzoyl peroxide, tert-butyl peroxybenzoate, tert-butyl peroxyisopropyl-carbonate, 2,5-bis(2-ethylhexanoylperoxy)-2,5-dimethylhexane, tert-butyl peroxy-2-ethylhexanoate, tert-butyl peroxy-3,5,5-trimethylhexanoate, dicumyl peroxide, 1,1-bis(tert-butylperoxy)cyclohexane, 1,1-bis(tert-butylperoxy)-3,3,5-trimethylcyclohexane, cumyl hydroperoxide, tert-butyl hydroperoxide, bis(4-tert-butylcyclohexyl)peroxydicarbonate, mixtures of two or more of these specified compounds with one another, and mixtures of these specified compounds with unspecified compounds which can likewise form free radicals.

These compounds are often used in an amount of from 0.01 to 10% by weight, preferably from 0.5 to 3% by weight, based on the weight of the monomers.

In this connection, it is possible to use different poly(meth)acrylates which differ, for example, in their molecular weight or in the monomer composition.

Furthermore, the polymethyl methacrylate shaped bodies can comprise further polymers in order to modify the properties. These include, inter alia, polyacrylonitriles, polystyrenes, polyethers, polyesters, polycarbonates and polyvinyl chlorides. These polymers can be used individually or as a mixture, where also copolymers which can be derived from the abovementioned polymers.

The plastic substrates according to the invention can, for example, be prepared from moulding materials of the abovementioned polymers. In this connection, use is generally made of thermoplastic moulding processes, such as extrusion or injection moulding.

The weight-average of the molecular weight M_{w} of the homopolymers and/or copolymers to be used according to the invention as moulding material for the preparation of the plastic substrates can vary within wide ranges, the molecular weight usually being matched to the intended use and the processing method of the moulding material. However, it is generally in the range between 20 000 and 1 000 000 g/mol, preferably 50 000 to 500 000 g/mol and particularly preferably 80 000 to 300 000 g/mol, without being limited by this. This parameter can be determined, for example, by means of gel permeation chromatography.

Furthermore, the plastic substrates can be produced by casting chamber processes. In this, suitable (meth)acrylic mixtures are, for example, placed into a mould and polymerized. Such (meth)acrylic mixtures generally have the above-described (meth)acrylates, in particular methyl methacrylate.

Furthermore, the (meth)acrylic mixtures of the above-described copolymers and, particularly for adjusting the viscosity, polymers, in particular, poly(meth)acrylates, may be present.

The weight-average of the molecular weight M_{w} of the polymers which are prepared by casting chamber processes is generally higher than the molecular weight of polymers which are used in moulding materials. This gives rise to a number of known advantages. In general, the weight average of the molecular weight of polymers which are prepared by casting chamber processes is in the range from 500 000 to 10 000 000 g/mol, without being limited by this.

According to a particular embodiment of the present invention, the matrix of the polymethyl methacrylate shaped body has at least 70%, preferably at least 80%, and particularly preferably at least 90% by weight, based on the weight of the polymethyl methacrylate shaped body, of polymethyl methacrylate.

The polymethyl methacrylate shaped body comprises 0.005 to 0.4% by weight, in particular 0.005 to 0.1% by weight, preferably 0.01 to 0.04% by weight, based on the total weight of the polymethyl methacrylate shaped body, of at least one triazine compound according to formula (I)

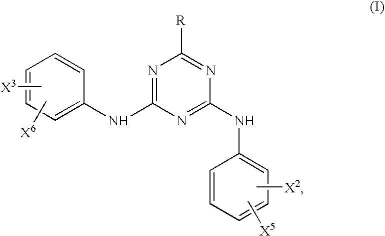

in which
R is halogen, for example fluorine, chlorine, bromine or iodine, C₁-C₂₂-alkyl, C_{I}-C₂₂-alkoxy, C_{I}-C₂₂-hydroxyalkoxy, C_{I}-C₂₂-alkoxyalkyl, —NHR₁—, —N(R¹)₂, or a radical of the formula (Ia) 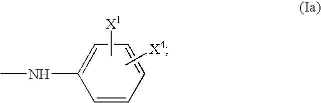
X¹, X² and X³,
   independently of one another, are —CONHR¹, —NR²R³, —SO₂R⁴, —CN, —COR⁵ or —COOR⁶, in which
   R¹ is hydrogen, C₁-C₂₂-alkyl, C₆-C₂₀-aryl, C₇-C₂₀-aralkyl, C₅-C₈-cycloalkyl, or a radical of the formula (Ib) 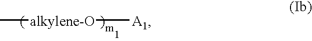
      in which
      A₁ is C_{I}-C₈-alkyl, C₅-C₆-cycloalkyl, C₆-C₂₀-aryl, C₇-C₂₀-aralkyl;
      and
      m₁is 1 to 10,
   R² and R³, independently of one another, are hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —COR¹;
   R⁴ is hydrogen, C_{I}-C₂₂-alkyl, C₆-C₈-cycloalkyl, C₆-C₂₀-aryl, C₇-C₂₀-aralkyl, a radical of the formula (Ib) or a radical of the formula —NR²R³;

R⁵ and R⁶, independently of one another, are hydrogen, C_{I}-C₂₂-alkyl, C₆-C₈-cycloalkyl, C₆-C₂₀-aryl, C₇-C₂₀-aralkyl or a radical of the formula (Ib);
X⁴, X⁵ and X⁶, independently of one another, are hydrogen or hydroxyl.

According to a particular aspect of the present invention, triazine compounds are used which can be represented by formula (II)

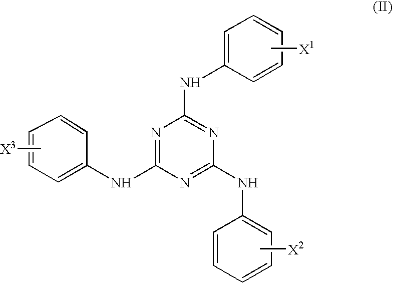

in which the radicals X¹, X², X³ have the meanings given above.

According to a particular embodiment of the present invention, triazine compound are used which can be represented by formula (III)

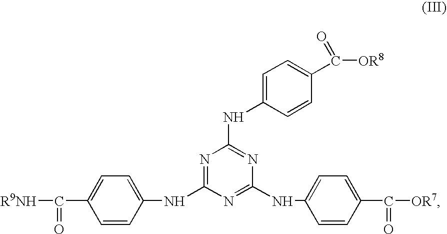

in which the radicals R⁷, R⁸ and R⁹, independently of one another, are C_{I}-C₂₂-alkyl.

The alkyl radicals according to the abovementioned formulae (I) to (III) may be linear or branched. Furthermore, these radicals may have substituents, for example halogen atoms. Preferred straight-chain and branched C_{I}-C₂₂-alkyl include, for example, methyl, ethyl, propyl, isopropyl, n-butyl, sec-butyl, isobutyl, t-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl or eicosyl.

The alkoxy radicals according to the abovementioned formulae (I) and (II) may be linear or branched. Furthermore, these radicals may have substituents, for example halogen atoms.

Examples of straight-chain and branched C_{I}-C₂₂-alkoxy are, for example, methoxy, ethoxy, propoxy, isopropoxy, butoxy, isobutoxy, sec-butoxy, tert-butoxy, pentoxy, isopentoxy, n-heptyloxy, n-octyloxy, isooctyloxy, n-nonyloxy, isononyloxy, decyloxy, n-dodecyloxy, heptadecyloxy octadecyloxy or eicosyloxy.

Preferred cycloalkyl groups include the cyclopropyl, cyclobutyl, cyclopentyl, cyclohexyl, cycloheptyl and the cyclooctyl group, which are optionally substituted by branched or unbranched alkyl groups.

Preferred cycloalkoxy groups include cycloalkoxy groups whose hydrocarbon radical is one of the abovementioned preferred cycloalkyl groups.

The aryl radicals may be optionally substituted by one or more C₁-C₄-alkyl or alkoxy radicals. Examples of C₆-C₂₀-aryl are, in particular, phenyl, naphthyl and biphenylyl.

The aralkyl radicals may be optionally substituted by one or more C₁— to C₄-alkyl or alkoxy radicals. Examples of C₇-C₂₀-aralkyl are benzyl, phenethyl, α-methylphenethyl or α,α-dimethylbenzyl.

“Alkylene” means a bivalent alkylene group having, preferably, 1 to 5, in particular 2 to 4, carbon atoms. These radicals include, inter alia, methylene, ethylene, n-propylene, isopropylene, n-butylene, isobutylene, pentylene, 2-ethylpropylene.

Particular preference is given to using the triazine compound which can be represented by formula (IV)

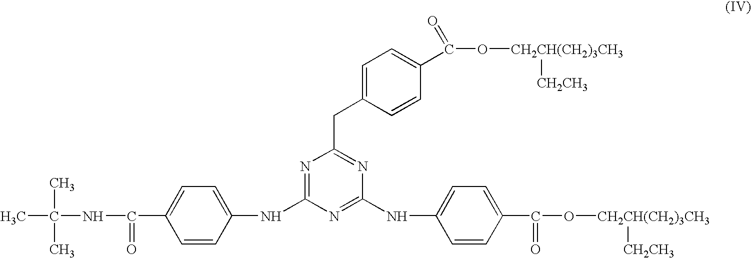

The compounds according to the above formulae and the preparation thereof are known per se and are described, for example, in EP-A 0 818 450.

The compound according to formula (IV), bis(2-ethylhexyl) 4,4′-[[6-[[4-[[1,1-dimethylethyl)-amino]carbonyl]phenyl]amino]-1,3,5-triazine-2,4-diyl]-diimino]bisbenzoacetate, can be obtained commercially from Ciba under the trade name ®Uvasorb HEB.

Furthermore, the polymethyl methacrylate shaped body can comprise further known additives, their amount, however, being restricted to the intended use of the tanning aids according to the invention. These include, inter alia, antistatics, antioxidants, mould-release agents, flame retardants, lubricants, dyes, flow improvers, fillers, light stabilizers, UV absorbers and organic phosphorus compounds, such as phosphites or phosphonates, pigments, anti-weathering agents and plasticizers.

Preferred additives include dyes which, dissolved in methyl methacrylate at a concentration of 0.01% by weight, display a transmission at 350 nm of at least 30%. Such dyes are known per se and are available, for example, under the trade name ®Makrolex blue RR, ®Makrolex violet B, ®Makrolex violet 3R, ®Makrolex green 5B, ®Makrolex green G, from Bayer, ®Sandoplast blue 2B, ®Sandoplast red BB, and ®Sandoplast green G from Clariant, ®Mikrolit violet BK from Ciba.

Furthermore, preferred additives include IR absorbers. These include, inter alia, IR-Absorber 2052 from Bayer.

Furthermore, the polymethyl methacrylate shaped body can comprise spherical particles. For the purposes of the present invention, the term spherical means that the particles preferably have a ball-like shape, it being obvious to the person skilled in the art that, due to the preparation methods, particles with a different shape may also be present, or that the shape of the particles can deviate from the ideal ball shape.

Accordingly, the term spherical means that the ratio of the largest dimension of the particles to the smallest dimension is at most 4, preferably at most 2, these dimensions in each case being measured through the centre of gravity of the particles. Preferably at least 70%, particularly preferably at least 90%, based on the number of particles, are sperical.

The particles preferably have an average diameter (weight-average) in the range from 5 to 50 μM, preferably in the range from 8 to 25 μm. More favourably, 75% of the particles are in the range from 5 to 35 μm.

These particles may, for example, be made of inorganic materials, in particular of BaSO₄ or plastic, preference being given to plastic particles. In this connection, the refractive index of the particles has a refractive number no measured at the Na-D line (589 nm) and at 20° C. which differs from the refractive number no of the matrix plastic by 0.02 to 0.2 unit.

The spherical plastic particles preferably comprise crosslinked polystyrene and/or crosslinked poly(meth)acrylates.

Mixtures from which the plastic particles are prepared particularly preferably have at least 80% by weight of styrene and at least 0.5% by weight of divinylbenzene.

The preparation of crosslinked plastic particles is known in the specialist field. For example, the scatter particles can be prepared by emulsion polymerization, as described, for example, in EP-A 342 283 or EP-A 269 324, very particularly preferably by polymerization in organic phase, as described, for example, in German Patent Application P 43 27 464.1, where, in the case of the last-mentioned polymerization technique, particularly narrow particle size distributions or, put another way, particularly small deviations of the particle diameters from the average particle diameter, arise.

The polymethyl methacrylate shaped body can comprise, for example, 2 to 50% by weight, preferably 4 to 10% by weight, of particles which develop a scattering effect.

As a result of this, a reduction in the dazzling effect by solar irradiation can surprisingly be achieved without tanning of the skin being impaired too much. This preferred embodiment of the tanning aid according to the invention is particularly suitable for applications in which a dazzling effect of the sun is to be reduced.

According to a particular aspect of the present invention, the polymethyl methacrylate shaped body has an impact strength of at least 20 kJ/m² measured in accordance with DIN 53453 (standard test piece). To improve this mechanical property, known impact-resistance modifiers in particular can be used.

Preferred impact-resistant moulding materials which can be used to produce the polymethyl methacrylate shaped body comprise 1 to 30, preferably 2 to 20, particularly preferably 3 to 15, in particular 5 to 12, % by weight of an impact-resistance modifying agent, which represents an elastomer phase of crosslinked polymer particles.

The impact-resistance modifying agent can be obtained in a manner known per se by bead polymerization or by emulsion polymerization.

Preferred impact-resistance modifying agents represent crosslinked particles with an average particle size in the range from 50 to 1 000 nm, preferably 60 to 500 nm and particularly preferably 80 to 120 nm.

Particles of this type can be obtained, for example, by the free-radical polymerization of mixtures which generally at least 40% by weight, preferably 50 to 70% by weight, of methyl methacrylate, 20 to 80% by weight, preferably 25 to 35% by weight, of butyl acrylate, and 0.1 to 2% by weight, preferably 0.5 to 1% by weight, of a crosslinking monomer, e.g. a multifunctional (meth)acrylate, such as, for example, allyl methacrylate and comonomers which can be copolymerized with the abovementioned vinyl compounds.

The preferred comonomers include, inter alia, C₁-C₄-alkyl(meth)acrylates, such as ethyl acrylate or butyl methacrylate, preferably methyl acrylate, or other vinylically polymerizable monomers, such as, for example, styrene. The mixtures for the preparation of the abovementioned particles may preferably comprise 0 to 10% by weight, preferably 0.5 to 5% by weight, of comonomers.

Particularly preferred impact strength modifying agents are polymer particles which have a two-layer, particularly preferably a three-layer, core-shell construction. Such core-shell polymers are described, inter alia, in EP-A 0 113 924, EP-A 0 552 351, EP-A 0 465 049 and EP-A 0 683 028.

Particularly preferred impact strength modifiers based on acrylate rubber have, inter alia, the following construction:
Core: Polymer with a methyl methacrylate fraction of at least 90% by weight, based on the weight of the core.
Shell 1: Polymer with a butyl acrylate fraction of at least 80% by weight, based on the weight of the first shell.
Shell 2: Polymer with a methyl methacrylate fraction of at least 90% by weight, based on the weight of the second shell.

As well as comprising the specified monomers, the core and the shells may in each case comprise further monomers. These have been described above, particularly preferred comonomers having a crosslinking effect.

For example, a preferred acrylate rubber modifier may have the following construction:
Core: Copolymer of methyl methacrylate (95.7% by weight), ethyl acrylate (4% by weight) and allyl methacrylate (0.3% by weight)
S1: Copolymer of butyl acrylate (81.2% by weight), styrene (17.5% by weight) and allyl methacrylate (1.3% by weight)
S2: Copolymer of methyl methacrylate (96% by weight) and ethyl acrylate (4% by weight)

The ratio of core to shell(s) in the acrylate rubber modifier may vary within wide ranges. The weight ratio of core to shell C/S is preferably in the range from 20:80 to 80:20, preferably from 30:70 to 70:30 to modifiers with one shell, and the ratio of core to shell 1 to shell 2 C/S1/S2 is in the range from 10:80:10 to 40:20:40, particularly preferably from 20:60:20 to 30:40:30 in the case of modifiers with two shells.

The particle size of the core-shell modifiers is usually in the range from 50 to 1 000 nm, preferably 100 to 500 nm and particularly preferably from 150 to 450 nm, without being limited thereby.

According to a particular embodiment, the polymethyl methacrylate shaped body has a E modulus of at least 2 800 N/mm², preferably at least 3 300 N/mm² according to ISO 527/2.

The thickness of the polymethyl methacrylate shaped body can be within a wide range depending on the intended use. In general, this shaped body has a to 20 mm.

The surface of the tanning aids may appear shiny or matt. According to a particular embodiment, the tanning aids may be equipped with a satin surface.

The size of the tanning aid can be adapted to the requirements. Thus, for example, roofs may be several 100 m² in size or smaller screens may comprise nearly 1 to 2 m².

The invention is illustrated in more detail by Example 1 below without the invention being limited to this example.

### EXAMPLE 1

A polymethyl methacrylate panel with a thickness of 3 mm was produced in a casting process. For this, 994.208 g of syrup (mixture of methyl methacrylate/polymethyl methacrylate with 8 to 10% conversion), 0.039 g of ®Mikrolit violet BK, 0.03 g of Solvaperm red BB, 0.200 g of ®Uvasorb HEB obtainable from Ciba Geigy and 0.55 g of 2,2-azodi(isobutyronitrile) (AIBN, from Akzo Nobel were mixed and poured into a casting glass mould. This mould was heated at 77° C. for 150 minutes.

The panel obtained in this way had a UV transmission at 380 nm of more than 60% and at 330 nm a transmission of less than 0.1%. The calculated effective radiation intensity was 4.79 mW/m².

The effective irradiation intensity can be calculated by the erythema function, which is expressed the efficiency of ultraviolet radiation for producing reddening of human skin. The evaluation is carried out by multiplying the transmitted irradiation spectrum by the erythema function.

The irradiation spectrum arises from the solar spectrum in accordance with standard DIN 67501. Integration of the evaluated spectrum over the wavelength gives the effective irradiation intensity, where the effectiveness refers to the efficacy for producing sunburn. The original solar spectrum has an effective irradiation intensity of about 250.9 mW/m².

1-15. (canceled)

16. A tanning aid, characterized in that the tanning aid comprises a polymethyl methacrylate shaped body, where the transparency of the polymethyl methacrylate shaped body at 380 nm is at least 40%, and the polymethyl methacrylate shaped body comprises 0.005 to 0.4% by weight, based on the weight of the polymethyl methacrylate shaped body, of at least one triazine compound according to formula (I) 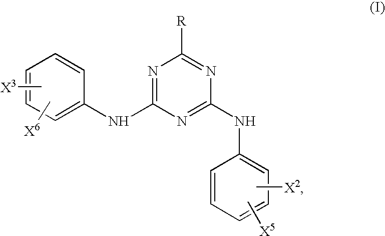
in which
R is halogen, C₁-C₂₂-alkyl, C₁-C₂₂-alkoxy, C_{I}-C₂₂-hydroxyalkoxy, C_{I}-C₂₂-alkoxyalkyl, —NHR₁—,
—N(R¹)₂, or a radical of the formula (Ia) 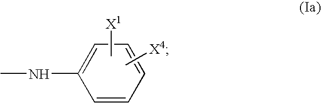
X¹, X² and X³, independently of one another, are —CONHR¹, —NR²R³, —SO₂R⁴, —CN, —COR⁵ or —COOR⁶;
R¹ is hydrogen, C₁-C₂₂-alkyl, C₆-C₂₀-aryl, C₇-C₂₀-aralkyl, C₅-C₈-cycloalkyl, or a radical of the formula (Ib) 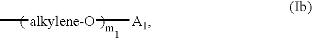
in which
A₁ is C₁-C₈-alkyl, C₅-C₆-cycloalkyl, C₆-C₂₀-aryl, C₇-C₂₀-aralkyl;
and
m₁ is 1 to 10,
R² and R³, independently of one another,
are hydrogen, C₁-C₂₂-alkyl, C₆-C₈-cycloalkyl, C₆-C₂₀-aryl, C₇-C₂₀-aralkyl, a radical of the formula (Ib) or a radical of the formula —COR¹;
R⁴ is hydrogen, C₁-C₂₂-alkyl, C₆-C₈-cycloalkyl, C₆-C₂₀-aryl, C₇-C₂₀-aralkyl, a radical of the formula (Ib) or a radical of the formula —NR²R³;
R⁵ and R⁶, independently of one another,
are hydrogen, C_{I}-C₂₂-alkyl, C₆-C₈-cycloalkyl, C₆-C₂₀-aryl, C₇-C₂₀-aralkyl or a radical of the formula (Ib);
X⁴, X⁵ and X⁶, independently of one another,
are hydrogen or hydroxyl.

17. The tanning aid according to claim 16, characterized in that the at least one triazine compound can be represented by formula (II) 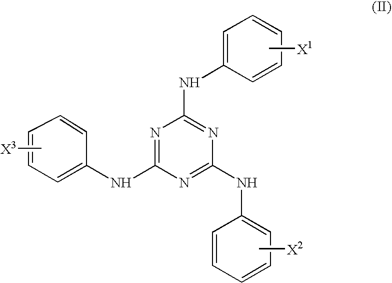
in which the radicals X¹, X², X³ have the meaning given in claim 16.

18. The tanning aid according to claim 16, characterized in that the at least one triazine compound can be represented by formula (III) 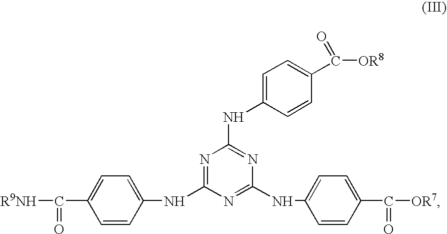
in which the radicals R⁷, R⁸ and R⁹, independently of one another, are C_{I}-C₂₂-alkyl.

19. The tanning aid according to claim 16, characterized in that the at least one triazine compound can be represented by formula (VI)

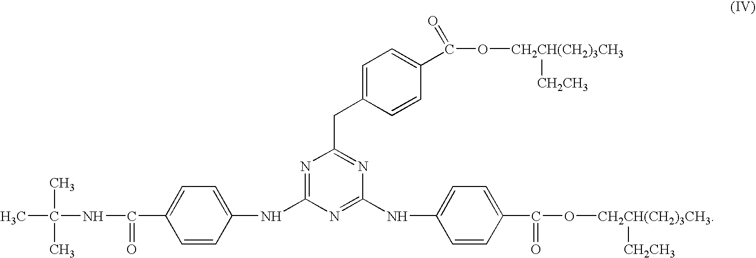

20. The tanning aid according to claim 16, characterized in that the tanning aid comprises 0.01 to 0.04% by weight, based on the weight of the polymethacrylate shaped body, of the at least one triazine compound.

21. The tanning aid according to claim 16, characterized in that the plastic shaped body additionally comprises dyes which, dissolved in MMA at a concentration of 0.01% by weight, exhibit a transmission of at least 30% at 350 nm.

22. The tanning aid according to claim 16, characterized in that the transparency of the polymethyl methacrylate shaped body at 400 nm is at least 30%.

23. The tanning aid according to claim 16, characterized in that the transparency of the polymethyl methacrylate shaped body at 330 nm is at most 30%.

24. The tanning aid according to claim 16, characterized in that the ratio of transparency of the polymethyl methacrylate shaped body at 400 nm to the transparency at 330 nm is at least 20.

25. The tanning aid according to claim 16, characterized in that the polymethyl methacrylate shaped body comprises an IR absorber.

26. The tanning aid according to claim 16, characterized in that the polymethyl methacrylate shaped body has an impact resistance of at least 20 kJ/m².

27. The tanning aid according to claim 16, characterized in that the polymethyl methacrylate shaped body has a thickness in the range from 1 to 200 mm.

28. The tanning aid according to claim 16, characterized in that the polymethyl methacrylate shaped body has an E modulus of at least 2,800 N/mm².

29. The tanning aid according to claim 16, characterized in that the polymethyl methacrylate shaped body comprises spherical particles.
